(19) United States
(12) Patent Application Publication
(10) Pub. No.: US 2006/0122233 A1
(43) Pub. Date: Jun. 8, 2006

(54) Rabeprazole calcium

(76) Inventors: Yatendra Kumar, Qutab Enclave (IN); Mohan Prasad, Enclave (IN); Neela Praveen Kumar, Malakpet (IN)

Correspondence Address: Jayadeep R Deshmukh;Ranbaxy Pharmaceuticals Inc, Suite 2100, 600 College Road East, Princeton, NJ, 08540, US

(21) Appl. No.: 10/539,681

(22) PCT Filed: Dec. 16, 2003
(86) PCT No.: PCT/IB03/05614

§ 371 Date: Jun. 16, 2005

(30) Foreign Application Priority Data
Dec. 16, 2002 (IN) 1265/DEL/2002

(51) Int. Cl. A61K 31/4439; C07D 403/02

(52) U.S. Cl. 514/338; 546/273.7

(57) ABSTRACT

The present invention relates to calcium salts of rabeprazole and processes for preparing rabeprazole calcium. The invention also relates to pharmaceutical compositions that include the rabeprazole calcium and use of said compositions for the treatment or prevention of gastrointestinal ulcers

### FIELD OF THE INVENTION

The field of the invention relates to calcium salts of rabeprazole and processes for preparing rabeprazole calcium. The invention also relates to pharmaceutical compositions that include the rabeprazole calcium and use of said compositions for the treatment or prevention of gastrointestinal ulcers.

### BACKGROUND OF THE INVENTION

Chemically, rabeprazole is 2-[[4-(3-methoxypropoxy)-3-methyl-2-pyridinyl]-methylsulfinyl]-1H-benzimidazole. Rabeprazole is a proton pump inhibitor and an antibacterial agent. Rabeprazole sodium is used for treating and preventing peptic ulcers, and for treating bacterial infections caused by camphylobacter and helicobacter pylori.

U.S. Pat. No. 5,045,552 discloses several substituted pyridylmethylsulfinyl benzimidazoles, including rabeprazole. It also discloses that some of these compounds can form salts with metals such as sodium, potassium, calcium or magnesium. However, only sodium salts of the disclosed compounds have been prepared. In particular, only the sodium salt of rabeprazole has been synthesized. Rabeprazole sodium is obtained in amorphous form by the process described in this patent, and is hygroscopic in nature.

A recent Japanese Patent Application JP 2001039975 describes non hygroscopic crystals of benzimidazolyl pyridylmethyl sulfoxides, including rabeprazole sodium, and their preparation. However, the inventors are not aware of any disclosure of a calcium salt of rabeprazole in the prior art. It is known that different salts and morphs of biologically active compounds may have different absorption profile in vivo and consequently different pharmacokinetic profile.

### SUMMARY OF THE INVENTION

In one general aspect there is provided calcium salts of rabeprazole i.e. rabeprazole calcium. In particular, there is provided rabeprazole hemicalcium salt.

In another general aspect there is provided rabeprazole calcium in a crystalline form.

The crystalline form of rabeprazole calcium may have the X-ray diffraction pattern of FIG. 1, the infrared spectrum of FIG. 2 and the differential scanning calorimetry peaks of FIG. 3.

In another general aspect there is provided a pharmaceutical composition that includes a therapeutically effective amount of a crystalline form of rabeprazole calcium; and one or more pharmaceutically acceptable carriers, excipients or diluents.

In another general aspect there is provided rabeprazole calcium in a substantially amorphous form.

The amorphous form of rabeprazole calcium may have the X-ray diffraction pattern of FIG. 4, the infrared spectrum of FIG. 5 and the differential scanning calorimetry peak of FIG. 6.

In another general aspect there is provided a pharmaceutical composition that includes a therapeutically effective amount of an amorphous form of rabeprazole calcium; and one or more pharmaceutically acceptable carriers, excipients or diluents.

In another general aspect there is provided a process for the preparation of the rabeprazole calcium. The process includes contacting rabeprazole free base or rabeprazole sodium with a calcium salt of an acid in one or more solvents; and isolating the rabeprazole calcium from the solution thereof by the removal of the solvent.

In another aspect, the process may be carried out in the presence of a base if rabeprazole free base is used as the starting material.

The solvent may be one or more of water, lower alkanol, ketone, ester, ether, nitrile, hydrocarbon, dipolar aprotic solvent, or mixtures thereof. The lower alkanol may include one or more of primary, secondary and tertiary alcohol having from one to six carbon atoms. The lower alkanol may include one or more of methanol, ethanol, denatured spirit, n-propanol, isopropanol, n-butanol, isobutanol, and t-butanol. In particular, the lower alkanol may include one or more of methanol, ethanol, n-propanol and isopropanol.

The ketone may include one or more of acetone, 2-butanone, and 4-methylpentan-2-one. The ester may include one or more of methyl acetate, ethyl acetate and isopropyl acetate. The ether may include one or more of dioxane and tetrahydrofuran. The nitrile may include, for example acetonitrile. The dipolar aprotic solvent may include one or more of dimethylsulfoxide and dimethylformamide. The hydrocarbon may include one or more of hexane and toluene

The calcium salt of an acid may be one or more of salt of an inorganic or organic acid. The calcium salt of an inorganic acid may include one or more of calcium chloride, calcium nitrate, calcium sulphate, calcium phosphate, calcium carbonate, and calcium dihydrogenphosphate. The calcium salt of an organic acid may include one or more of calcium oxalate, calcium acetate, calcium lactate, calcium succinate, calcium citrate, and calcium tartrate.

The base which may be used along with rabeprazole free base may include one or more of alkali metal hydroxide, alkali metal carbonate or alkali metal bicarbonate. The alkali metal hydroxide may include one or more of sodium hydroxide and potassium hydroxide. The alkali metal carbonate may include one or more of sodium carbonate and potassium carbonate. The alkali metal bicarbonate may include sodium bicarbonate. Isolating the rabeprazole calcium may include one or more of filtration, filtration under vacuum, decantation and centrifugation. The process may include further forming of the product so obtained into a finished dosage form.

The process may include further drying of the product obtained from the solution.

In another general aspect there is provided a method of treating or preventing gastrointestinal ulcers using therapeutically effective amount of the rabeprazole calcium.

The details of one or more embodiments of the inventions are set forth in the description below. Other features, objects and advantages of the inventions will be apparent from the description and claims.

### DESCRIPTION OF THE DRAWINGS

FIG. 1 is X-ray powder diffraction pattern of crystalline form of rabeprazole calcium prepared as described herein.

FIG. 2 is an infrared spectrum in KBr of crystalline form of rabeprazole calcium.

FIG. 3 is differential scanning calorimetry peaks of crystalline form of rabeprazole calcium.

FIG. 4 is X-ray powder diffraction pattern of an amorphous form of rabeprazole calcium prepared as described herein.

FIG. 5 is an infrared spectrum in KBr of an amorphous form of rabeprazole calcium.

FIG. 6 is differential scanning calorimetry peak of an amorphous form of rabeprazole calcium.

### DETAILED DESCRIPTION OF THE INVENTION

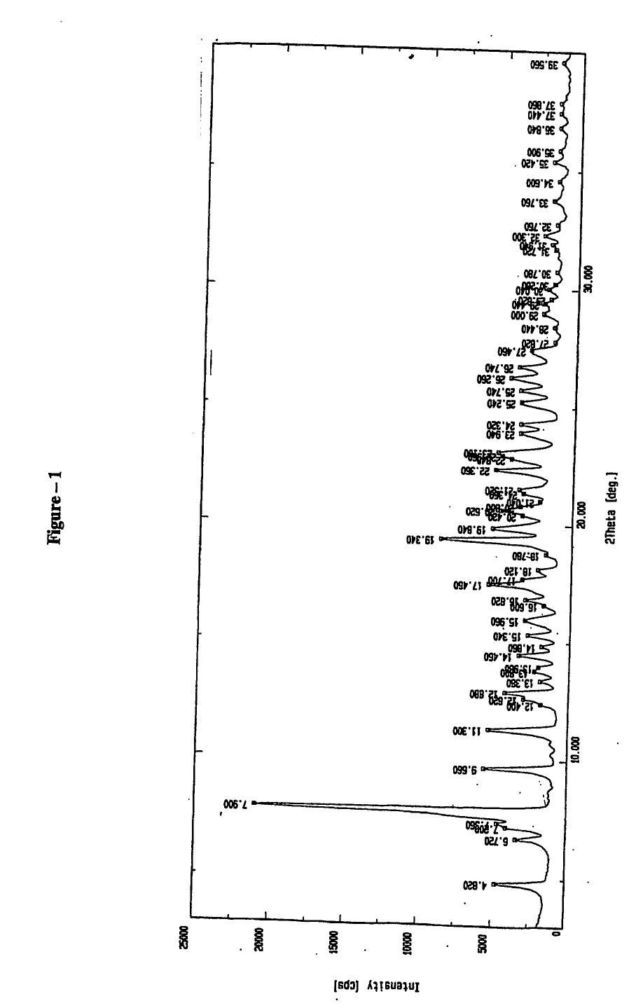

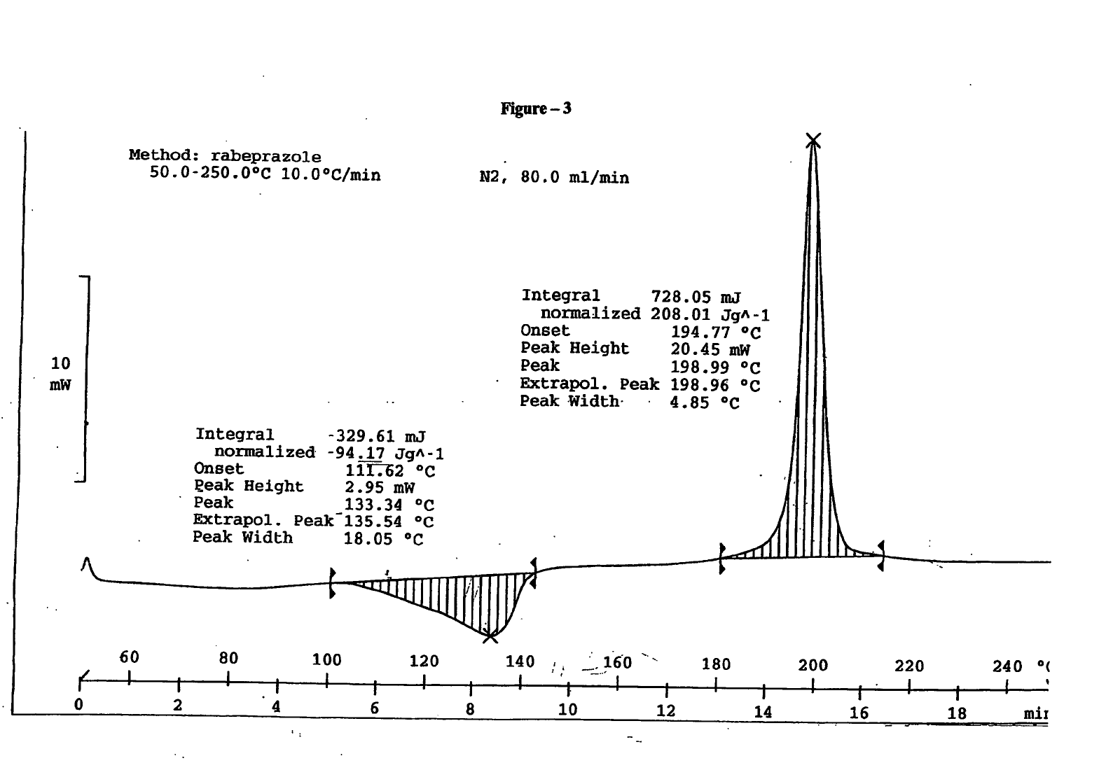

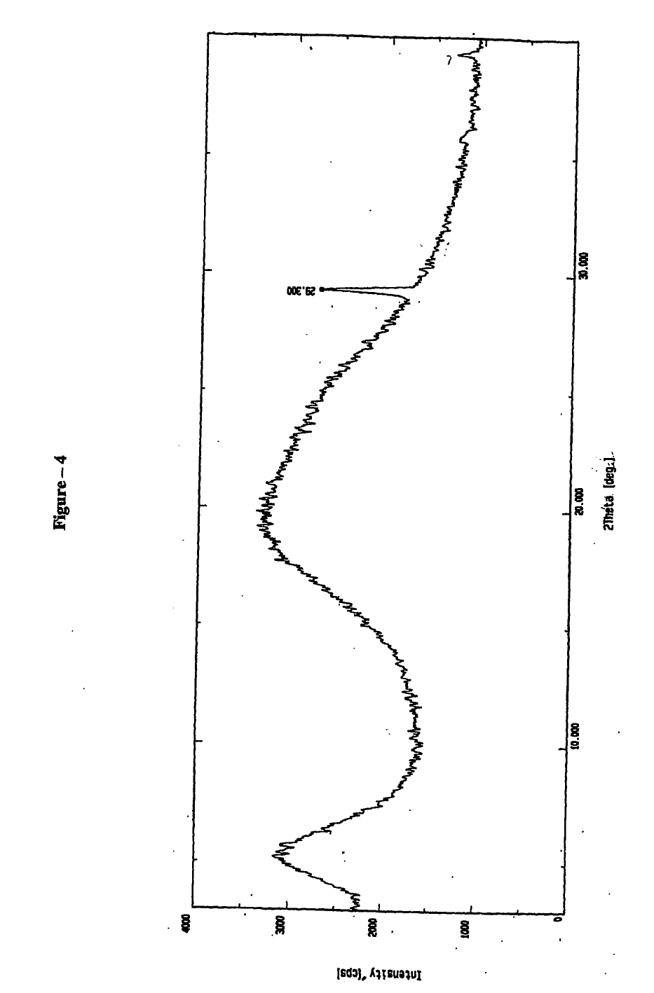

The inventors have found a new salt of rabeprazole, the calcium salt of rabeprazole and, in particular, the rabeprazole hemicalcium. The rabeprazole calcium has been found in both crystalline and amorphous forms. The crystalline rabeprazole calcium is characterized by its X-ray powder diffraction pattern, infrared spectrum and differential scanning calorimetry as shown in FIGS. 1, 2 and 3, respectively. The amorphous rabeprazole calcium is characterized by its X-ray powder diffraction pattern, infrared spectrum and differential scanning calorimetry as shown in FIGS. 4, 5 and 6, respectively. The inventors also have developed a process for the preparation of the crystalline and amorphous forms of rabeprazole calcium, by contacting the rabeprazole free base or rabeprazole sodium with a calcium salt of an acid in one or more solvents; and isolating the rabeprazole calcium from the solution thereof by the removal of the solvent.

The inventors also have developed pharmaceutical compositions that contain the crystalline and amorphous forms of the rabeprazole calcium, in admixture with one or more solid or liquid pharmaceutical diluents, carriers, and/or excipients. These pharmaceutical compositions may be used for the treatment or prevention of gastrointestinal ulcers.

The term “rabeprazole calcium” includes any salt comprised of rabeprazole anions and calcium cations, including, for example, solid as well as dissolved forms, crystalline and amorphous forms.

Further, the term “rabeprazole calcium” encompasses stoichiometric as well as non-stoichiometric ratios of rabeprazole anion and calcium cation, including, for example, the ratio of rabeprazole to calcium to be 1:1, 1:2 or 2:1. In particular, the rabeprazole calcium salt can be formed having a 2:1 molar ratio between rabeprazole anion and calcium cation, for example, rabeprazole hemicalcium. The rabeprazole hemicalcium can be formed even when an excess of rabeprazole or an excess of calcium salt of an acid is used in the salt formation.

The crystalline and substantially amorphous forms of rabeprazole calcium can be obtained in forms which are non hygroscopic. The amorphous form may be advantageous in that it is obtained as a finely powdered form with better solubility properties.

The rabeprazole calcium and, in particular the rabeprazole hemicalcium can exist in an anhydrous and/or solvent-free form, or as a hydrate and/or a solvate. The hydrates and alcohol solvates of rabeprazole calcium and, in particular the hydrates and alcohol solvates of rabeprazole hemicalcium, form another aspect of the invention. In particular, methanol solvate of rabeprazole hemicalcium forms another aspect of the invention.

Further, the rabeprazole calcium can exist as one of the two enantiomers due to the presence of a chiral center. The enantiomers may either be separated, for example by subjecting the rabeprazole free base or the sodium salt to resolution using an optical purity embedding agent (CN 1223262, see chem. Abs. 133:17460) and converted to the corresponding calcium salt, or prepared by a stereo selective oxidation of the corresponding sulfide in the presence of a chiral titanium complex and a base (U.S. Pat. No. 5,948,789), and converted to the corresponding calcium salt The individual enantiomers as well as the mixtures thereof are likewise all embraced by the singular expression “rabeprazole calcium.”

In general, the rabeprazole calcium may be prepared by contacting rabeprazole free base or its sodium salt, with a calcium salt of an acid in a suitable solvent. In general, a solution may be obtained by dissolving the calcium salt of an acid and rabeprazole free base or its sodium salt in a suitable solvent. If rabeprazole free base is used as a starting material, the reaction may be carried out in the presence of a base.

In General, the rabeprazole calcium may be precipitated out of the solution or reaction mixture. The precipitation may be spontaneous depending upon the solvent and the conditions used. Alternatively, the precipitation can be induced by reducing the temperature of the solution. The precipitation may also be facilitated by reducing the volume of the solution or by adding an antisolvent, i.e. a solvent in which the rabeprazole calcium is insoluble or sparingly soluble.

The rabeprazole free base or its sodium salt can be obtained by methods known in the art, for example U.S. Pat. Nos. 6,313,303 and 5,948,789; WO 01/04109, 02/062786, and WO 02/083608.

The term “suitable solvent” includes any solvent or solvent mixture in which rabeprazole base or its sodium salt, is soluble, including, for example, water, lower alkanol, ketone, ester, ether, nitrile, hydrocarbon, dipolar aprotic solvent, and mixtures thereof. Examples of lower alkanol include those primary, secondary and tertiary alcohols having from one to six carbon atoms. Suitable lower alkanol solvents include methanol, ethanol, denatured spirit, n-propanol, isopropanol, n-butanol, isobutanol, and t-butanol. A suitable lower alkanol includes one or more of methanol, ethanol, n-propanol and isopropanol.

Examples of ketones include solvents such as acetone, 2-butanone, and 4-methylpentan-2-one. Examples of esters include solvents such as methyl acetate, ethyl acetate and isopropyl acetate. Examples of ethers include dioxane and tetrahydrofuran. Examples of nitriles include acetonitrile. A suitable dipolar aprotic solvent includes one or more of dimethylsulfoxide and dimethylformamide. Examples of hydrocarbons include solvents such as hexane and toluene. Mixtures of all of these solvents are also contemplated.

The calcium salt of an acid can be a salt of an inorganic or organic acid. The calcium salt of an inorganic acid includes one or more of calcium chloride, calcium nitrate, calcium sulphate, calcium phosphate, calcium carbonate, and calcium dihydrogenphosphate. The calcium salt of an organic acid includes one or more of calcium oxalate, calcium acetate, calcium lactate, calcium succinate, calcium citrate, and calcium tartrate.

If rabeprazole base is used as a starting material, a base of alkali metal hydroxide, alkali metal carbonate or alkali metal bicarbonate may be used. Examples of alkali metal hydroxides include sodium hydroxide and potassium hydroxide. Examples of alkali metal carbonates include sodium carbonate and potassium carbonate. Suitable alkali metal bicarbonate includes sodium bicarbonate.

The precipitated calcium salt can be isolated in a solid state by conventional methods such as filtration, filtration under vacuum, decantation or centrifugation. The rabeprazole calcium can be washed with a suitable solvent. It may also be further purified by crystallization in an appropriate solvent, for example water, an alcohol such as methanol, or a ketone such as acetone.

The product obtained may be further or additionally dried to achieve the desired moisture values. For example, the product may be further or additionally dried in a tray drier, dried under vacuum and/or in a Fluid Bed Drier.

The resulting rabeprazole calcium may be formulated into ordinary dosage forms such as, for example, tablets, capsules, pills, solutions, etc. In these cases, the medicaments can be prepared by conventional methods with conventional pharmaceutical excipients.

The compositions include dosage forms suitable for oral, buccal, rectal, and parenteral (including subcutaneous, intramuscular, and ophthalmic) administration. The oral dosage forms may include solid dosage forms, like powder, tablets, capsules, suppositories, sachets, troches and lozenges as well as liquid suspensions, emulsions, pastes and elixirs. Parenteral dosage forms may include intravenous infusions, sterile solutions for intramuscular, subcutaneous or intravenous administration, dry powders to be reconstituted with sterile water for parenteral administration, and the like.

Further, the rabeprazole calcium dosage forms described herein can be used in a method for treatment or prevention of gastrointestinal ulcers. The method of treatment includes administering to a mammal in need of treatment a dosage form that includes a therapeutically effective amount of the rabeprazole calcium.

Rabeprazole calcium is a useful proton pump inhibitor and an antibacterial, and thus can be used to treat any condition that would be benefited by administration of a gastric acid secretion inhibitor. In particular, rabeprazole calcium can be used for healing of erosive or ulcerative gastroesophageal reflux disease (GERD); maintenance of healing of erosive or ulcerative GERD; healing of duodenal ulcer; treatment of pathological hypersecretory conditions, including Zollinger-Ellison Syndrome, by administering an effective amount of the salt to a patient in need thereof. The specific form of rabeprazole calcium to be used is not particularly limited and specifically includes rabeprazole hemicalcium.

The present invention is further illustrated by the following examples which are provided merely to be exemplary of the invention and is not intended to limit the scope of the invention. Although the examples are directed to rabeprazole calcium, the principles described in these examples can be applied to other salts of rabeprazole.

Preparation of Rabeprazole Calcium in Crystalline Form

### EXAMPLE 1

### 2-[[4-(3-methoxypropoxy)-3-methyl-2-pyridinyl]-methylsulfinyl]-1H-benzimidazole, hemicalcium salt, methanol solvate

Sodium hydroxide flakes (2.8 g, 0.07 m) were dissolved in methanol (175 ml). To the above solution rabeprazole base (25 g, 0.0696 m) was added at 15-20° C. and stirred for 15 minutes. Calcium acetate (7.7 g, 0.048 m) was added to the resulting solution. The reaction mixture was stirred for 30 minutes and the solution was filtered to remove the undissolved particles. The filtrate was stirred for about 14 hours at room temperature, and the separated solid was filtered. The solid was washed with methanol and dried under vacuum at 40° C. to give white crystalline rabeprazole calcium (23.2 g).

Assay (by HPLC): 99.0%, Water (w/w): 7.56%, Ca content (w/w): 5.41%

1H-HMR (DMSO-d6, δ, ppm); 1.94-2.02(m, 2H), 2.08(s, 3H), 3.18(s, 3H), 3.25(s, 3H), 3.51(t, 2H), 4.09-4.13(t, 2H), 4.47(dd, 1H), 4.65(dd, 1H), 6.88-6.97(m, 3H), 7.49-7.52(m, 2H), 8.30(d, 1H).

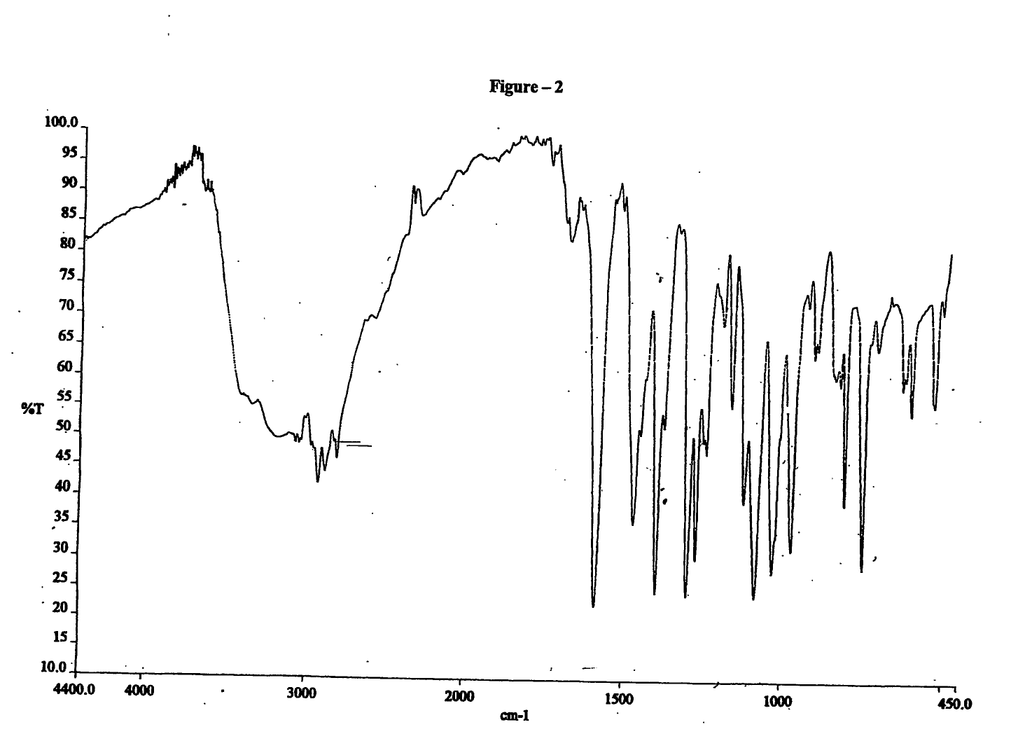

XRD, IR and DSC spectra are as shown in FIGS. 1, 2, & 3, respectively.

### EXAMPLE 2

### 2-[[4-(3-methoxypropoxy)-3-methyl-2-pyridinyl]-methylsulfinyl]-1H-benzimidazole, hemicalcium salt, methanol solvate

Rabeprazole sodium (25 g, 0.0656 m) was dissolved in methanol (175 ml) at room temperature. Calcium acetate (7.7 g, 0.048 m) was added to the above solution. The clear solution was stirred for 14 hours at room temperature; the solid that separated out was filtered, washed with methanol and dried under vacuum at 40° C. to give ²3.0 g of white crystalline rabeprazole calcium.

Assay (by BPLC): 99.03%, Water (w/w): 4.93%.

XRD, IR, NMR and DSC spectra are similar to those for example 1.

Preparation of Rabeprazole Calcium in Amorphous Form

### EXAMPLE 3

### 2-[[4-(3-methoxypropoxy)-3-methyl-2-pyridinyl]-methylsulfinyl]-1H-benzimidazole, hemicalcium salt

Rabeprazole sodium (25 g, 0.0656 m) was dissolved in water (100 ml) at room temperature. Calcium acetate (7.7 g, 0.048 m) dissolved in water (25 ml) was slowly added to the above solution. Rabeprazole calcium precipitated out simultaneously. The suspension was further stirred for 30 minutes; the obtained solid was filtered and washed with water. The product was dried under reduced pressure at 40° C. to give rabeprazole calcium (22.6 g).

Assay (by HPLC): 99.5%, Water (w/w): 6.93%, Ca content (w/w): 6.16%

X-ray powder diffraction pattern (FIG. 4) showed a plain halo, which demonstrates the amorphous nature of the product.

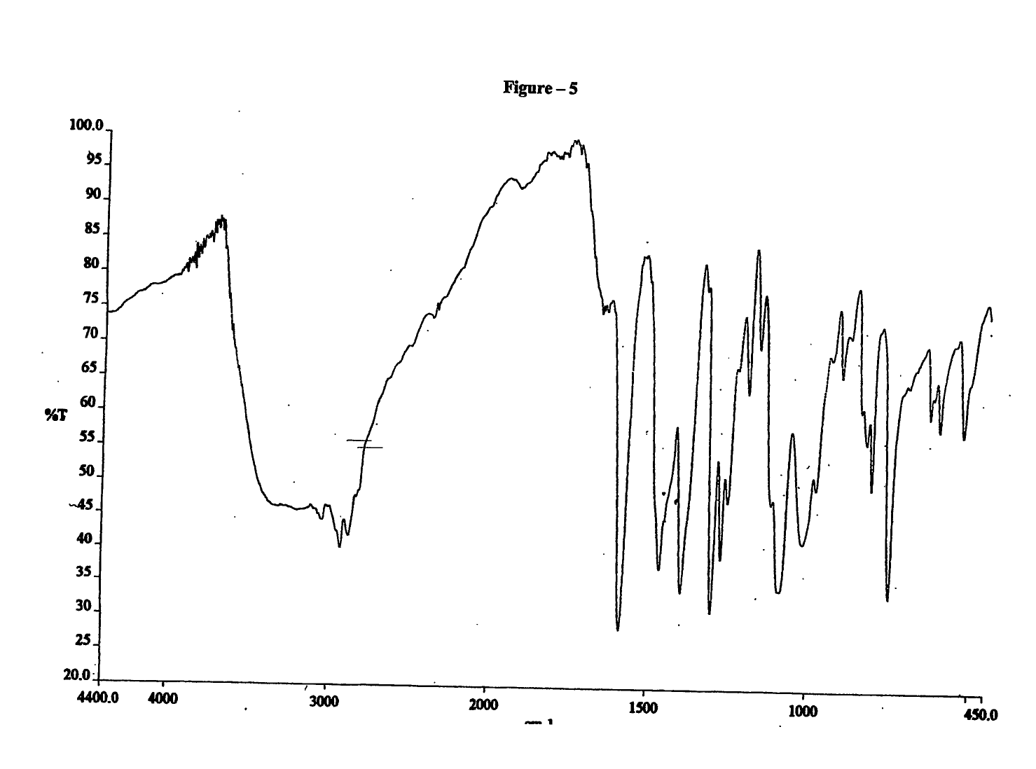

Infrared spectrum in KBr (FIG. 5) is different than one obtained for crystalline form of rabeprazole calcium.

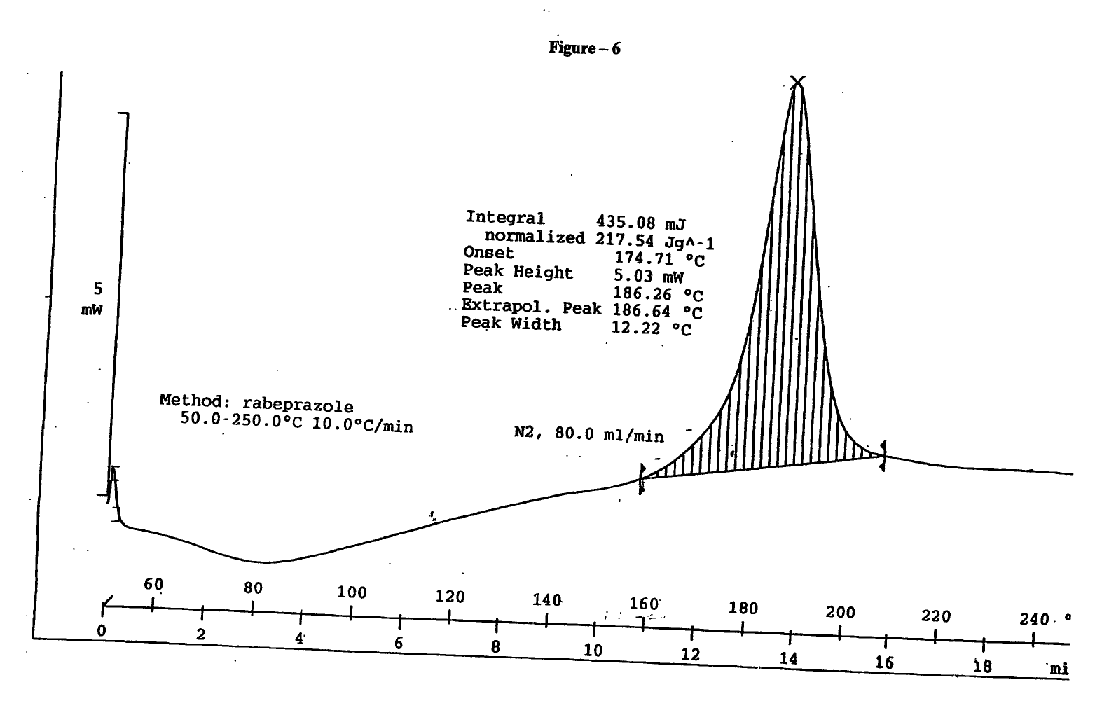

Differential scanning calorimetry (FIG. 6) is different than one obtained for crystalline form of rabeprazole calcium.

### EXAMPLE 4

### 2-[[4-(3-methoxypropoxy)-3-methyl-2-pyridinyl]-methylsulfinyl]-1H-benzimidazole, hemicalcium salt

Rabeprazole sodium (20 g, 0.052) was dissolved in water (100 ml), and a solution of calcium chloride (3.0 g, 0.27 m) in water (25 ml) was added slowly to it. The reaction mixture was stirred for one hour. The separated solid was filtered and washed with water. The product was dried under vacuum at 40° C. to yield rabeprazole calcium (19 g).

Assay (by HPLC): 98.1%, Water (w/w): 7.41%, Ca content (w/w): 6.06%

XRD, IR and DSC spectra are similar to those for example 3.

While the present invention has been described in terms of its specific embodiments, certain modifications and equivalents will be apparent to those skilled in the art and are intended to be included within the scope of the present invention.

1. A calcium salt of rabeprazole.

2. The salt of claim 1, which is rabeprazole hemicalcium.

3. The salt of claim 1, which is in a crystalline form.

4. The salt of claim 3, which is an alcohol solvate.

5. The salt of claim 4, which is a methanol solvate.

6. The salt of claim 1, which is in a substantially amorphous form.

7. The salt of claim 1, which is hydrated.

8. The salt of claim 3, wherein the rabeprazole calcium has the X-ray diffraction pattern of FIG. 1.

9. The salt of claim 3, wherein the rabeprazole calcium has the infrared spectrum of FIG. 2.

10. The salt of claim 6, wherein the rabeprazole calcium has the X-ray diffraction pattern of FIG. 4.

11. The salt of claim 6, wherein the rabeprazole calcium has the infrared spectrum of FIG. 5.

12. A pharmaceutical composition comprising:
a therapeutically effective amount of rabeprazole calcium; and one or more pharmaceutically acceptable carriers, excipients or diluents.

13. A process for the preparation of rabeprazole calcium, the process comprising:
contacting rabeprazole free base or rabeprazole sodium with a calcium salt of an acid in a suitable solvent; and
isolating the rabeprazole calcium from the solution thereof by the removal of the solvent.

14. The process of claim 13, wherein the calcium salt of an acid is a salt of an inorganic acid.

15. The process of claim 14, wherein the calcium salt comprises one or more of calcium chloride, calcium nitrate, calcium sulphate, calcium phosphate, calcium carbonate, and calcium dihydrogenphosphate.

16. The process of claim 13, wherein the calcium salt of an acid is a salt of an organic acid.

17. The process of claim 16, wherein the calcium salt comprises one or more of calcium oxalate, calcium acetate, calcium lactate, calcium succinate, calcium citrate, and calcium tartrate.

18. The process of claim 13, wherein the solvent comprises one or more of water, lower alkanol, ketone, ester, ether, nitrile, hydrocarbon, dipolar aprotic solvent, or mixtures thereof.

19. The process of claim 18, wherein the lower alkanol comprises one or more of primary, secondary and tertiary alcohol having from one to six carbon atoms.

20.-27. (canceled)

26. The process of claim 13, further comprising adding a base if rabeprazole free base is used as a starting material.

27. The process of claim 28, wherein the base comprises one or more of an alkali metal hydroxide, alkali metal carbonate and alkali metal bicarbonate.

28. (canceled)

29. The process of claim 13, wherein the rabeprazole calcium precipitates out spontaneously from the solvent.

32. The process of claim 13, wherein removing the solvent comprises one or more of filtration, filtration under vacuum, decantation, and centrifugation.

33. The process of claim 13, wherein one or more of rabeprazole hemicalcium, a crystalline form of rabeprazole calcium, an alcohol solvate, a substantially amorphous form of rabeprazole calcium, or a hydrate of rabeprazole calcium is isolated from the solution.

34.-38. (canceled)

39. The process of claim 13, further comprising additional drying of the product obtained.

40. The process of claim 13, further comprising forming the product obtained into a finished dosage form.

41. The process of claim 13, wherein the rabeprazole calcium has the X-ray diffraction pattern of FIG. 1 or FIG. 4.

42. The process of claim 13, wherein the rabeprazole calcium has the infrared spectrum of FIG. 2 or FIG. 5.

43.-44. (canceled)

45. A method for treating or preventing gastrointestinal ulcers, which comprises administering to a patient in need thereof an effective amount of rabeprazole calcium.

46. The method of claim 45, wherein the rabeprazole calcium is used for healing of erosive or ulcerative gastroesophageal reflux disease (GERD); maintenance of healing of erosive or ulcerative GERD; healing of duodenal ulcer; or treatment of pathological hypersecretory conditions, including Zollinger-Ellison Syndrome.

47. The method of claim 45, wherein the rabeprazole calcium comprises rabeprazole hemicalcium.

48. A The pharmaceutical composition of claim 12, wherein the pharmaceutical composition is intended for use in the treatment or prevention of gastrointestinal ulcers.

49. The pharmaceutical composition of claim 12, wherein the rabeprazole calcium is rabeprazole hemicalcium.

50. The pharmaceutical composition of claim 12, wherein a crystalline form of the rabeprazole calcium is used.

51. The pharmaceutical composition of claim 12, wherein an alcohol solvate of the rabeprazole calcium is used.

52. The pharmaceutical composition of claim 12, wherein a substantially amorphous form of the rabeprazole calcium is used.

53. The pharmaceutical composition of claim 12, wherein a hydrate of the rabeprazole calcium is used.
